(19) United States
(12) Plant Patent Application Publication
(10) Pub. No.: US 2023/0189677 P1
(43) Pub. Date: Jun. 15, 2023

(54) CANNABIS PLANT NAMED 'DKJ127'

(50) ;

(71) Applicant: Vertanical GmbH, Gräfelfing (DE)

(72) Inventor: Bastian Baasch, Utting am Ammersee (DE)

(21) Appl. No.: 17/803,816

(22) Filed: Dec. 8, 2022

(30) Foreign Application Priority Data
Dec. 9, 2021 (QZ) 2021/3223

(51) Int. Cl. A01H 6/28

(52) U.S. Cl. PLT/258

(57) ABSTRACT

‘DKJ127’ is a Cannabis sativa cultivar suitable for production of herbal medicinal products (standardized extract) having high to very high THC content, about 17-20% THC eq.

Latin name of the genus and species: The Latin name of the novel plant cultivar disclosed herein is Cannabis sativa (L.).

Variety denomination: This new and distinct cultivar of Cannabis sativa (L.) has been given the denomination ‘DKJ127’.

### STATEMENT OF PRIORITY

This application claims the benefit, under 35 U.S.C. § 119 (a-c), of European Community Plant Variety Application No. 2021/3223 filed on Dec. 9, 2021, the entire contents of which is incorporated by reference herein.

### BACKGROUND OF THE INVENTION

Cannabis sativa is indigenous to Central Asia, and South Asia and has long been used for fiber (hemp), seed and seed oils, for medicinal purposes, and as a recreational drug. The present invention is directed to a new and distinct Cannabis sativa cultivar named ‘DKJ127’ having a high level of THC, a low degree of variation in chemical profile and low susceptibility to diseases, and which is suitable for further processing into a standardized medicinal product.

### Lineage, Cultivation, and Selection

A parental “motherplant” for ‘DKJ127’ was originally selected from seed of the Cannabis strain ‘Jack Herer’ using phenotype selection. Due to its complex lineage, seed of the ‘Jack Herer’ strain is known to display many different phenotypes and does not produce uniform plants. Therefore, phenotype selection was initiated to develop a suitable parental motherplant having the desirable phenotype, which was then further propagated via cuttings.

Two hundred ‘Jack Here’ seeds were sown, and a stock of motherplants was created by taking cuttings from the plants grown from each of the 200 ‘Jack Here’ seeds. The selection criteria for the specific genetics and phenotypes of the seed grown plants included the successful germination of the seed (must germinate), successful cultivation of the motherplant through the vegetative and flowering phases, the rooting behaviour of cuttings (successful rooting), the resistance to diseases, such as grey mould, and a tetrahydrocannabinolic acid (THCA) content of at least 15%.

The motherplants start as cuttings in 4×4 cm rockwool cubes, which are then transplanted three weeks later into 20×20 cm rockwool cubes. The lifetime of a motherplant is between 8 and 12 months, after which it is discarded. During its lifetime, a motherplant can deliver cuttings every second week, starting from week 8 of its life. Every time after taking cuttings, the motherplants get trimmed and shaped. Cuttings are grown in an 18-hour day light cycle with a 6-hour night period. Total time in vegetative stage is 2 weeks.

To induce flowering, the period of light is shortened to 12 h/day and the nutrient recipe is switched to a higher potassium and phosphorus blend to promote development of the flowers. The plants are pruned about 10 days after starting induction for flowering. Pruning removes unnecessary leaves and branches from the lower side of the plant to allow light to reach all flowers. The flower buds of ‘DKJ127’ are larger than those of other phenotypes of ‘Jack Herer’. The period of flowering stage for J127 is about 9 weeks after which flowers are harvested. Starting from 30-40 cm height, the plants can reach up to 140 cm-150 cm in height, at the end of the flowering stage.

As part of the selection process, batch-to-batch consistency was analyzed based on the cannabinoid and terpene profile. ‘DKJ127’ contains THCA at a content of approximately 18% and this high level of THCA was observed batch to batch for ‘DKJ127’. Only small amounts of other cannabinoids are present in ‘DK-J127’, mainly Cannabigerol (CBG) and Cannabidiol (CBD) at a content of about 0.7% and 0.2%. No other cannabinoids are present in ‘DKJ127’ in significant amounts. The major terpenes found in ‘DK-J127’ include alpha-pinene, camphene, beta-pinene, beta-myrcene, d-limonene, linalool, beta-caryophyllene, alpha-humulene, nerolidol, guaiol, and alpha-bisabolol.

Table 1 and Table 2 below provide data used in the selection process.

TABLE 1
THCA and percent weight loss on drying
of 22 selected strains

| | Loss on drying | |
|---|---|---|
| Strain-ID | % (w/w) | THCA |
| DK-J120-000 | 5% | 23.80% |
| DK-J127-000 | 5% | 21.70% |
| DK-J019-000 | 5% | 21.60% |
| DK-J115-000 | 6% | 20.50% |
| DK-J064-000 | 5% | 20.30% |
| DK-J087-000 | 5% | 20.10% |
| DK-J002-000 | 5% | 19.60% |
| DK-J186-000 | 5% | 19.10% |
| DK-J140-000 | 5% | 18.20% |
| DK-J168-000 | 6% | 17.90% |
| DK-J176-000 | 5% | 17.90% |
| DK-J149-000 | 5% | 17.80% |
| DK-J105-000 | 6% | 17.60% |
| DK-J187-000 | 6% | 17.50% |
| DK-J039-000 | 7% | 17.40% |
| DK-J166-000 | 6% | 17.20% |
| DK-J093-000 | 6% | 16.80% |
| DK-J024-000 | 6% | 16.60% |
| DK-J085-000 | 7% | 16.50% |
| DK-J110-000 | 5% | 16.50% |
| DK-J033-000 | 5% | 15.70% |
| DK-J099-000 | 7% | 12.40% |

TABLE 2
Yield and cultivation behaviour and THC content

| | | | | | | matur- | | | | | |
|---|---|---|---|---|---|---|---|---|---|---|---|
| | | wt | wt | | seeds | ity | | | rooting | rooting | THC |
| Strain- | ht | wet | dry | Fac- | in | level | | rooting | test 1 | test 2 | eq. |
| ID | [cm] | [g] | [g] | tor | flowers | [1-10] | disease | time | [20] | [11] | [%] |
| DK- | 155 | 260 | 60.25 | 4.3 | no | 8 | no | 10-18 | 20 | 11 | 19.6% |
| J002 | | | | | | | | days | | | |
| DK- | 175 | 600 | 133.2 | 4.5 | no | 9 | no | 10-18 | 20 | 10 | NA |
| J003 | | | | | | | | days | | | |
| DK- | 165 | 440 | 74.8 | 5.9 | no | 7 | no | 10-18 | 19 | 11 | 21.6% |
| J019 | | | | | | | | days | | | |
| DK- | 176 | 740 | 142.35 | 5.2 | no | 8 | no | 10-18 | 6 | 7 | 16.6% |
| J024 | | | | | | | | days | | | |
| DK- | 180 | 480 | 105 | 4.6 | no | 7 | no | 10-18 | 20 | 11 | 15.7% |
| J033 | | | | | | | | days | | | |
| DK- | 180 | 1460 | 369.92 | 3.9 | yes | 8 | no | 10-18 | 17 | 11 | 17.4% |
| J039 | | | | | | | | days | | | |
| DK- | 200 | 860 | 193.6 | 4.4 | no | 9 | no | 10-18 | 20 | 11 | 20.3% |
| J064 | | | | | | | | days | | | |
| DK- | 165 | 620 | 136.8 | 4.5 | no | 8 | no | 10-18 | 20 | 11 | 16.5% |
| J085 | | | | | | | | days | | | |
| DK- | 175 | 580 | 131.89 | 4.4 | no | 9 | no | 10-18 | 20 | 11 | 20.1% |
| J087 | | | | | | | | days | | | |
| DK- | 150 | 220 | 43.63 | 5.0 | no | 7 | no | 10-18 | 6 | 11 | NA |
| J088 | | | | | | | | days | | | |
| DK- | 155 | 2000 | 417.31 | 4.8 | no | 7 | no | 10-18 | 5 | 11 | 16.8% |
| J093 | | | | | | | | days | | | |
| DK- | 170 | 500 | 107.79 | 4.6 | no | 7 | no | 10-18 | 20 | 11 | 12.4% |
| J099 | | | | | | | | days | | | |
| DK- | 170 | 1040 | 216.14 | 4.8 | no | 8 | no | 10-18 | 20 | 11 | 17.6% |
| J105 | | | | | | | | days | | | |
| DK- | 130 | 1220 | 205.13 | 5.9 | no | 7 | no | 10-18 | 10 | 11 | 16.5% |
| J110 | | | | | | | | days | | | |
| DK- | 200 | 1020 | 212.83 | 4.8 | no | 9 | no | 10-18 | 20 | 11 | 20.5% |
| J115 | | | | | | | | days | | | |
| DK- | 180 | 840 | 193.27 | 4.3 | no | 9 | no | 10-18 | 19 | 11 | 21.7% |
| J127 | | | | | | | | days | | | |
| DK- | 160 | 1120 | 214.79 | 5.2 | no | 7 | grey | 10-18 | 10 | 11 | NA |
| J130 | | | | | | | mould | days | | | |
| DK- | 170 | 620 | 146.22 | 4.2 | no | 8 | no | 10-18 | 20 | 11 | 18.2% |
| J140 | | | | | | | | days | | | |
| DK- | 200 | 840 | 186.4 | 4.5 | no | 7 | no | 10-18 | 5 | 11 | 17.8% |
| J149 | | | | | | | | days | | | |
| DK- | 180 | 420 | 98.35 | 4.3 | no | 8 | no | 10-18 | 20 | 11 | 17.2% |
| J166 | | | | | | | | days | | | |
| DK- | 184 | 1120 | 236.56 | 4.7 | no | 8 | no | 10-18 | 20 | 11 | 19.1% |
| J186 | | | | | | | | days | | | |
| DK- | 185 | 1060 | 286.38 | 3.7 | no | 8 | no | 10-18 | 10 | 11 | 17.5% |
| J187 | | | | | | | | days | | | |

In summary, out of 200 ‘Jack Herer’ seedlings, 22 were selected for further analysis and asexual reproduction via cuttings. These 22 phenotypes were brought into the flowering phase and material of those was dried and analyzed for THCA content. The content of THCA, terpene profile and odour were included as criteria in the selection of the line named

J127 (DK-J127-000) (‘DKJ127’).

Asexual Reproduction. ‘DKJ127’ has been propagated as vegetative stem cuttings since its original selection. The characteristics disclosed herein for ‘DKJ127’ have remained stable and the plant has reproduced true to type through successive generations of asexual propagation.

### SUMMARY OF THE INVENTION

‘DK-J12’ is a high THC producing Cannabis cultivar. The flowers of ‘DKJ127’ are larger than the parent strain ‘Jack Herer’.

As used herein, the term “variety” is interchangeable with the terms “cultivar,” “line” and/or “strain.”

The following trait has been repeatedly observed and are determined to be the unique characteristics of ‘DKJ127’. This characteristic distinguishes ‘DKJ127’ as a new and distinct Cannabis cultivar:

1. High to very high THC content, about 17-20% THC eq. and absent to low CBD content, about 0.07% CBD eq.

### BRIEF DESCRIPTION OF THE DRAWINGS

The photographs in the drawings were made using conventional techniques and show the colors as true as reasonably possible by conventional photography. Colors in the photographs may differ slightly from the color values cited in the detailed botanical description, which accurately describe the colors of the new Cannabis sativa variety ‘DKJ127’.

FIG. 1 is a color photograph of a leaflet of the new cultivar ‘DKJ127’.

FIG. 2 is a color photograph of a stem of the new cultivar ‘DKJ127’.

FIG. 3 is a color photograph of a plant of the new cultivar ‘DKJ127’.

FIGS. 4A and 4B show color photographs of the new cultivar ‘DKJ127’ at different stages of growth. FIG. 3A shows a 5-month-old plant and FIG. 4B shows a 7-month-old plant.

FIG. 5 is a color photograph of a cutting of the new cultivar ‘DKJ127’, which is 1.5 weeks old.

FIG. 6 is a color photograph of a plant of the new cultivar ‘DKJ127’ in the vegetative stage that is 4.5 weeks old.

FIG. 7 is a color photograph of the stem of a plant of the new cultivar ‘DKJ127’ that is 7 months old.

FIG. 8 and FIG. 9 are a color photographs of the flower of a plant of the new cultivar ‘DKJ127’.

FIG. 10 shows the concentrations of various terpenes from different batches of ‘DKJ127’. ‘VER-01-THC’ is the commercial name for ‘DKJ127’.

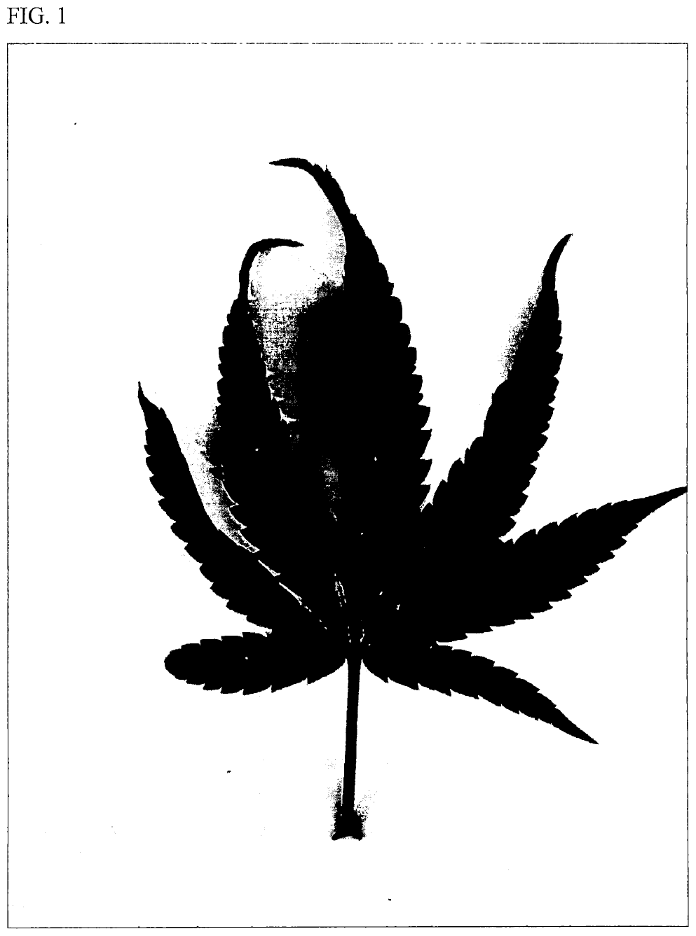

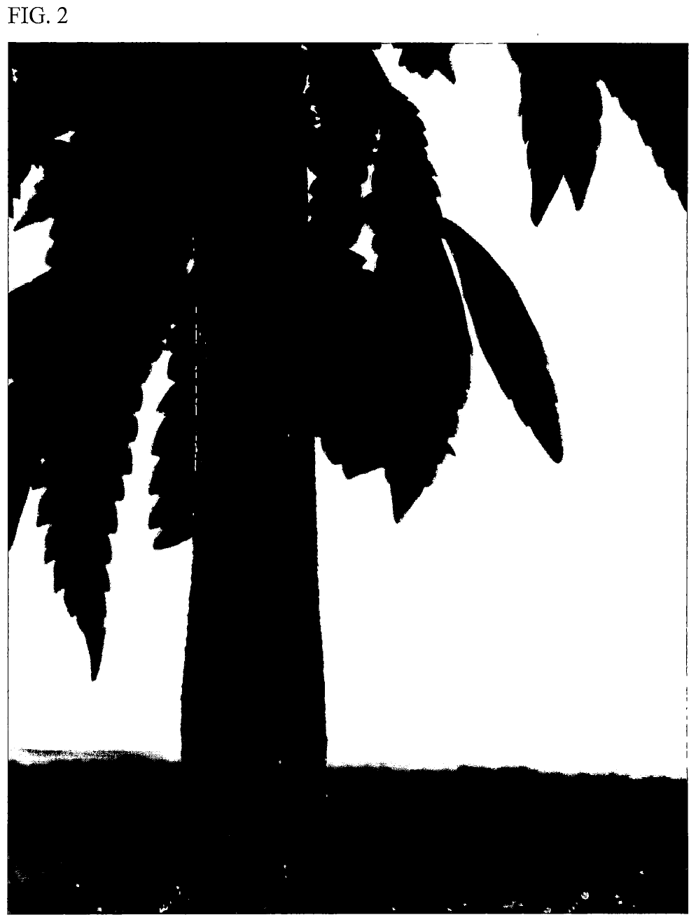

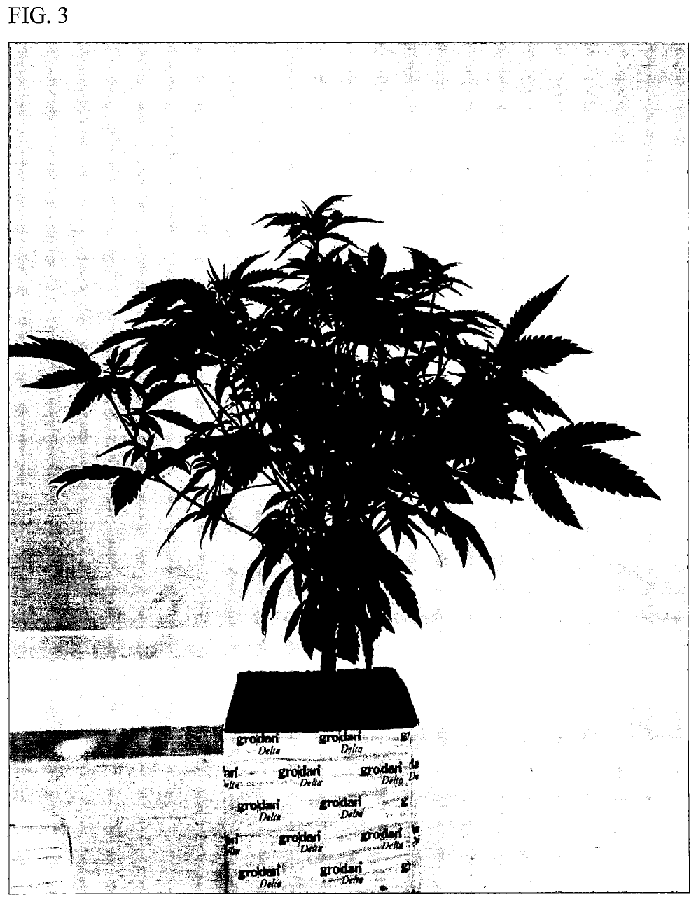

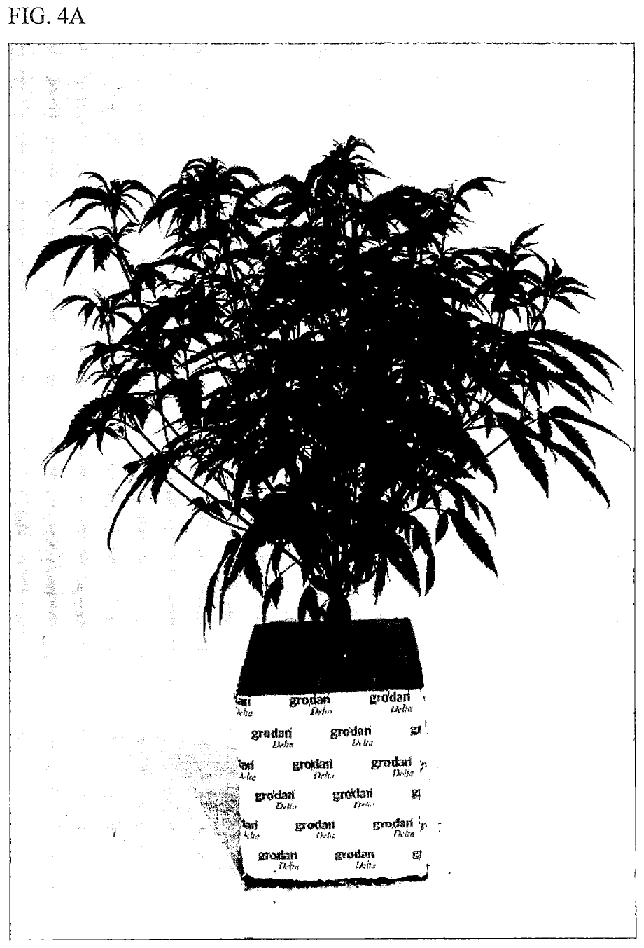

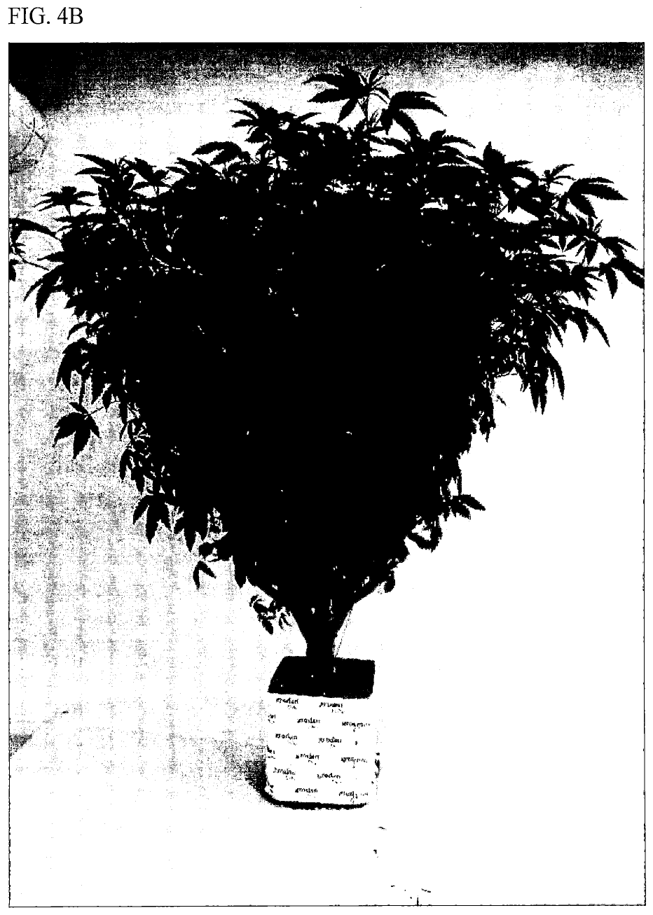

### DETAILED BOTANICAL DESCRIPTION

The following is a detailed description of the botanical characteristics of a new and distinct cultivar of Cannabis sativa plant given the designation ‘DKJ127’. Where dimensions, sizes, colors, and other characteristics are given, it is to be understood that such characteristics are approximations or averages set forth as accurately as practicable.

‘DKJ127’ has not been observed under all possible environmental conditions; therefore, the phenotype may vary under different environmental conditions such as season, temperature, light intensity, day length, cultural conditions, and the like, without, however, any variance in the genotype.

### TECHNICAL DESCRIPTION OF THE CULTIVAR

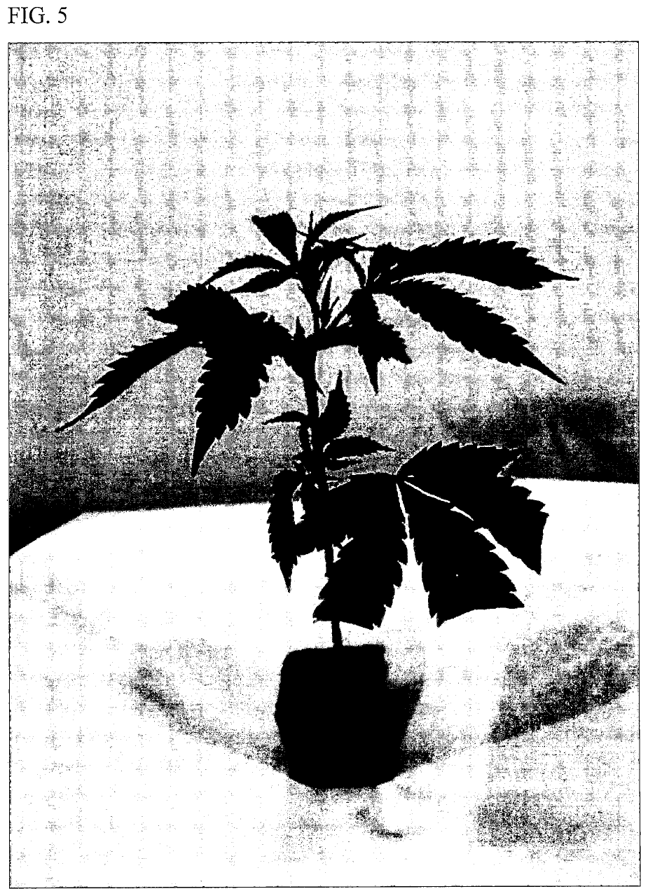

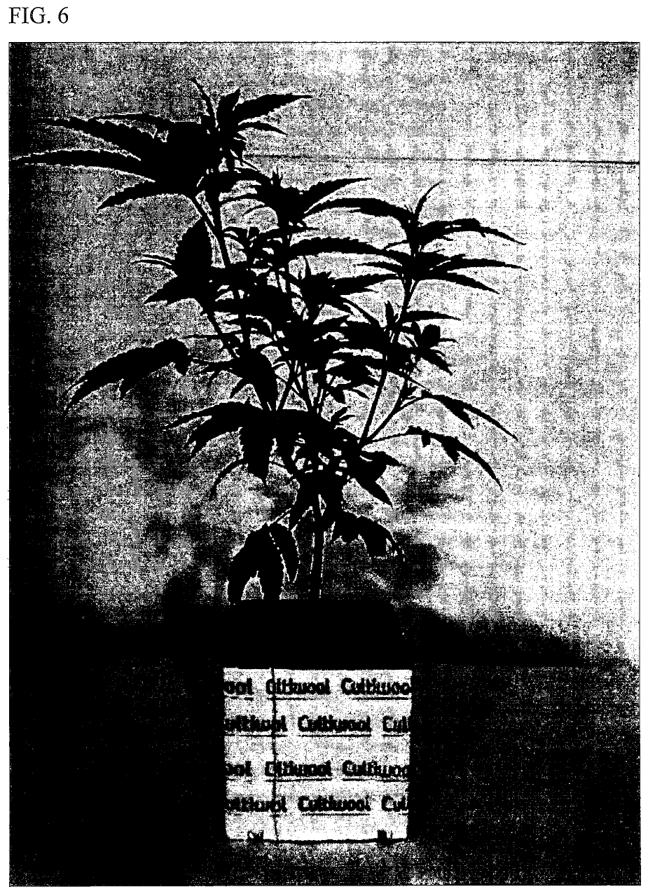

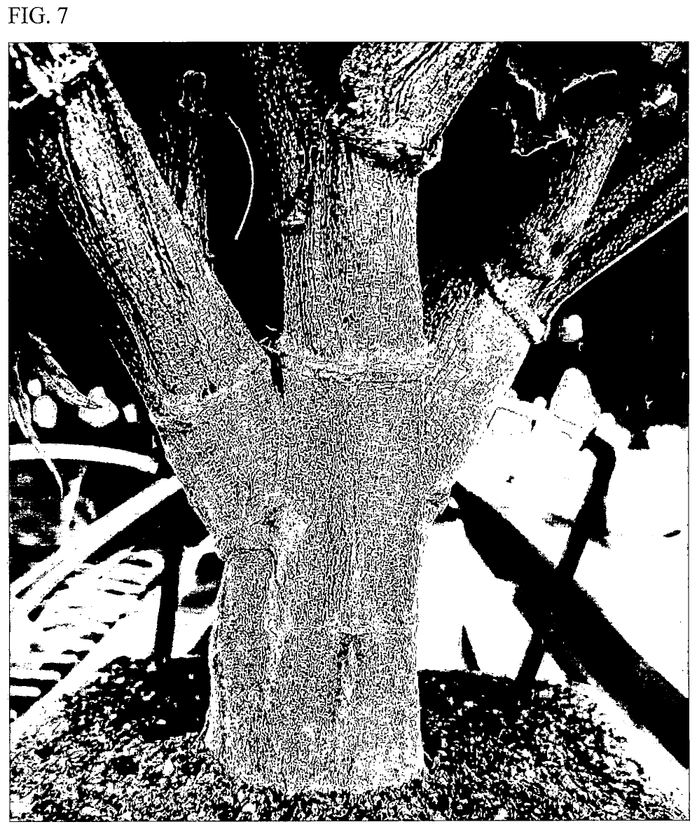

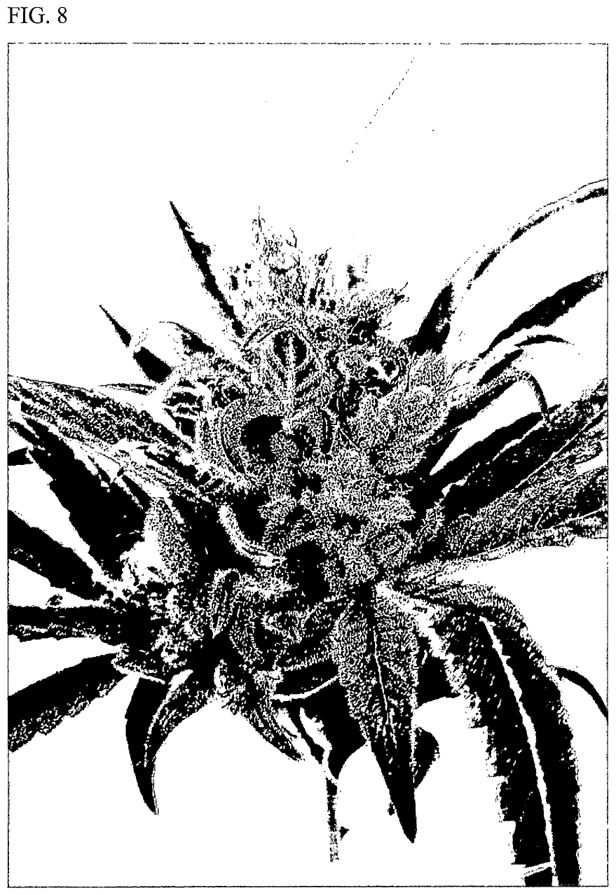

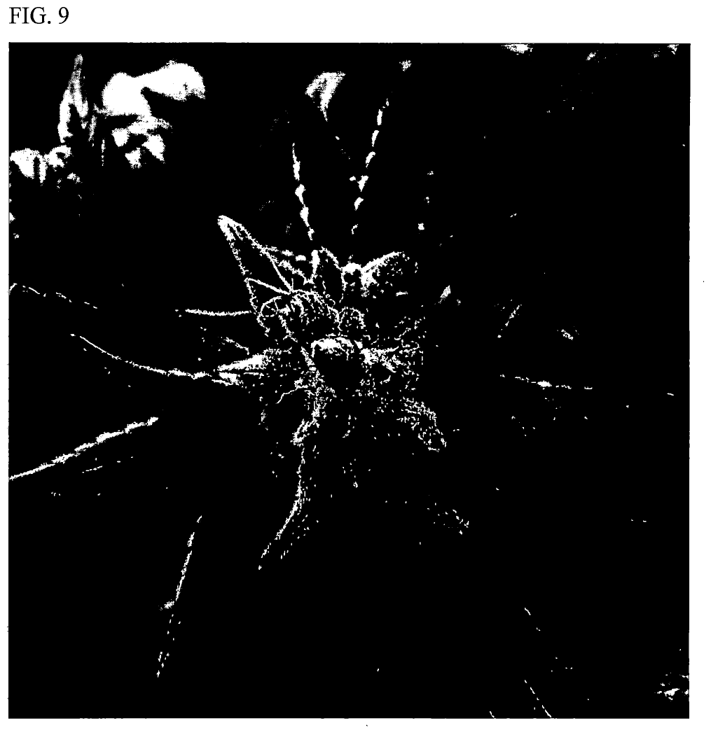

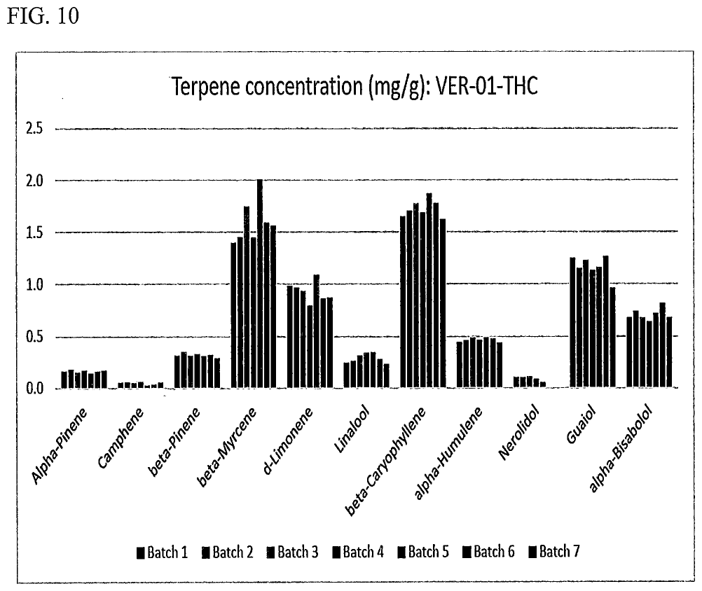

Classification: Botanical name: Cannabis sativa (L.). Common name: Cannabis . Variety name: ‘DKJ127’.
   Parentage.—Seedling selection grown from ‘Jack Herer’ seed.
   Propagation.—Typically propagated asexually via stem cuttings.
   Plant description.—Plant height: medium; about 140 cm-150 cm by the end of flowering.
   Foliage.—Leaf description: Number of leaflets: medium, similar to ‘Epsilon 68’. Central leaflet length: medium, similar to ‘Dioca 88’.
   Flower description.—Timing of female flowering: early to medium. Period of flowering: about 9 weeks. Color: dark to bright green, sometimes purple on the upper part.
   Chemical content.—THC and CBD content: about 17-20% THC eq. and about 0.07% CBD eq. Terpene content: DKJ127 produces alpha-pinene, camphene, beta-pinene, beta-myrcene, d-limonene, linalool, beta-caryophyllene, alpha-humulene, nerolidol, guaiol, and alpha-bisabolol. See, FIG. 5
Comparison to ‘Jack Herer’: ‘DKJ127’ has larger flower buds than those of other ‘Jack Herer’ phenotypes and consistently higher THC content, about 17-20% THC eq.

We claim:

1. A new and distinct cultivar of Cannabis sativa plant named ‘DKJ127’, substantially as illustrated and described herein.
